(19) United States
(12) Patent Application Publication
(10) Pub. No.: US 2024/0189229 A1
(43) Pub. Date: Jun. 13, 2024

(54) LIQUID PHARMACEUTICAL COMPOSITIONS OF BACLOFEN FOR ORAL ADMINISTRATION

(71) Applicant: ANI Pharmaceuticals, Inc., Baudette, MN (US)

(72) Inventors: Sushant Omprakash Dube, Navi Mumbai (IN); Suprit Dilip Saoji, Buldhana (IN); Girish G. Kore, Solapur (IN); Abhijit Balaji Pawar, Hadolti (IN); Purushottam Sakhahari Pattewar, Hyderabad (IN); Pankaj Kisan Chatki, Hyderabad (IN); Sumitra Ashokkumar Pillai, Hyderabad (IN); Praveen Kumar Subbappa, Princeton, NJ (US)

(73) Assignee: ANI Pharmaceuticals, Inc., Baudette, MN (US)

(21) Appl. No.: 18/114,502

(22) Filed: Feb. 27, 2023

(30) Foreign Application Priority Data
Oct. 19, 2022 (IN) 202241059722

(51) Int. Cl. A61K 9/08; A61K 9/00; A61K 31/197; A61K 47/38

(52) U.S. Cl. CPC A61K 9/08; A61K 9/0053; A61K 31/197; A61K 47/38

(57) ABSTRACT

Liquid pharmaceutical compositions of baclofen or a pharmaceutically acceptable salt thereof are described. More specifically, stable liquid pharmaceutical compositions of baclofen at concentrations of 2 mg/mL or more are provided. Preferably, the liquid pharmaceutical compositions are suitable for oral administration, and stable at pH ranges of 3-8 over a variety of storage conditions, including long-term storage for extended periods of time. Various methods for preparing stable liquid pharmaceutical compositions of baclofen are described. Methods of treating spasticity using the inventive pharmaceutical compositions are also provided.

### CROSS-REFERENCE TO RELATED APPLICATIONS

This application claims foreign priority to Indian Application No. IN 202241059722, filed on Oct. 19, 2022, which is incorporated herein by reference in its entirety.

### FIELD OF THE INVENTION

The present invention relates to liquid pharmaceutical compositions of baclofen or a pharmaceutically acceptable salt thereof.

### BACKGROUND OF THE INVENTION

Baclofen is a structural analog of the inhibitory neurotransmitter gamma-aminobutyric acid (GABA) and may exert its effects by stimulation of the GABA receptor subtype. Chemically, baclofen is 4-amino-3-(4-chlorophenyl)-butanoic acid, a derivative of γ-aminobutyric acid. It is represented by the following formula:

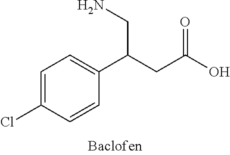 [SMILES: NCC(CC(=O)O)c1ccc(Cl)cc1]

Baclofen is a white to off-white, odorless or practically odorless crystalline powder, with a molecular weight of 213.66 g/mole. It is slightly soluble in water, very slightly soluble in methanol, and insoluble in chloroform. Its low solubility in water makes it difficult to obtain stable aqueous solutions of baclofen with concentrations greater than 2 mg/mL, and specifically greater than or equal to 5 mg/mL.

Presently, baclofen is approved in the United States under the brand names KEMSTRO® (oral disintegrating tablet), LIORESAL® (oral uncoated tablet & solution for intrathecal injection), OZOBAX® (oral solution; 1 mg/mL), LYVISPAH® (granules for oral suspension), and FLEQSUVY® (oral suspension; 5 mg/mL) for the alleviation of signs and symptoms of spasticity resulting from multiple sclerosis, particularly for the relief of flexor spasms and concomitant pain, clonus, and muscular rigidity. Further, these baclofen products may also be of some value in patients with spinal cord injuries and other spinal cord diseases.

The approved package insert for OZOBAX® (baclofen oral solution; 1 mg/mL) recommends that the maximum recommended dosage for OZOBAX® is 80 mg daily (20 mg four times a day). As a result, approximately 80 mL of OZOBAX® needs to be administered per day in order to achieve the maximum recommended dosage of baclofen. Spasticity in conditions such as multiple sclerosis or spinal cord injury is a chronic condition, requiring large volumes of baclofen solution to be administered, resulting in poor patient compliance. Therefore, it would be useful to have a higher concentration liquid pharmaceutical composition in order to reduce the overall volume required for oral administration of therapeutic dose, and to improve patient compliance and convenience.

FLEQSUVY® (oral suspension; 5 mg/mL) and extemporaneously compounded oral suspensions in the hospital pharmacies have certain drawbacks. Sedimentation of solids is a common problem which is encountered in oral suspension products, which leads to caking (formation of compact mass), making it difficult to dispense the suspension. An overdose of baclofen in patients may cause coma or progressive drowsiness, light-headedness, dizziness, somnolence, accommodation disorders, respiratory depression, seizures, or hypotonia progressing to loss of consciousness.

Administration of solid oral dosage forms to pediatric and geriatric population remains a challenge, particularly due to a frequent lack of age-appropriate formulations, which raises a concern about dose accuracy. Moreover, several factors specific to the pediatric and geriatric population, such as the ability to swallow, palatability issues, etc., may hamper the administration of oral medication. Indeed, most commercially available medicines are designed for adults and do not provide ease of use for pediatric and geriatric population. Beyond the efforts of health authorities to promote the development of pediatric and geriatric medicines, many medicinal products are authorized only for adults and are not currently available in formulations suitable for administration to pediatric and geriatric population. Consequently, caregivers or parents frequently modify medicines used off-label before administration in children, leading to dose error risk or inaccurate dosing, as well as stability and/or bioavailability issues pertaining to the drug.

It has been reported that baclofen could be dissolved in strongly acidic or strongly basic pH solutions. For example, Ahuja S., “Baclofen,” Analytical Profiles of Drug Substances, vol. 14, New York: Academic Press, pp. 527-548, 1985 (Ahuja 1985) reported that concentrations of baclofen greater than 20 mg/mL could be obtained by dissolving baclofen in aqueous solutions of 0.1N HCl or aqueous solutions of 0.1N NaOH. However, at a very high and a very low pH, baclofen undergoes alkaline and acidic hydrolysis, respectively, and forms baclofen-related Compound A which is listed in the USP monograph as a known impurity (BRC-A) that must be controlled to within certain limits.

Various sources have reported stable suspensions or syrups of baclofen for oral administration that had concentrations higher than an equilibrium concentration of 4.3 mg/mL (Allen L. V., et al., “Stability of Baclofen, Captopril, Diltiazem Hydrochloride, Dipyridamole, and Flecainide Acetate in Extemporaneously Compounded Oral Liquids,” Am J Health-Syst. Pharm., 53:2179-2184, 1996; and Johnson C. E., et al., “Stability of an extemporaneously compounded baclofen oral liquid,” Am. J. Hosp. Pharm., 50:2353-55, 1993), but none of these preparations were acceptable for pharmaceutical uses.

U.S. Pat. No. 8,357,379 discloses aqueous solutions comprising baclofen in concentrations greater than 2 mg/mL up to about 10 mg/mL. These solutions were prepared by heat and sonication; high speed stirring; alkalization with back titration; acidification with back titration.

U.S. Pat. No. 9,655,968 discloses aqueous solutions comprising baclofen in multivalent physiological ion solution such as artificial cerebrospinal fluid at concentrations of 10 mg/mL. The aqueous solutions disclosed in the above patents contains normal saline or multivalent physiological ion solution as solvents which are preferred diluents for injectable dosage forms and not preferred for preparing oral dosage forms.

U.S. Pat. No. 10,610,502 discloses aqueous oral solution comprising baclofen and a buffer comprising citric acid, a salt of citric acid, or any combination thereof. Further, it also discloses buffer free aqueous oral solutions of baclofen. This patent discloses oral solutions having a baclofen concentration of 1 mg/mL to 2 mg/mL.

Despite many attempts to design an aqueous solution of baclofen, there is no commercial ready-to-use solution formulation of baclofen with concentrations of 5 mg/mL or more meeting the requirements of health regulatory authorities.

What is needed is a method for providing a high-concentration, ready-to-use or ready-to-administer, stable pharmaceutical solution of baclofen for oral administration, without requiring use of strong acids or strong bases for dissolving baclofen, and without utilizing sonication and intense agitation to dissolve the baclofen. There also appears to be a clinical need for providing concentrated aqueous solutions of baclofen for oral administration, having acceptable pharmaceutical properties, preferably, concentrated solutions that are stable in a variety of storage conditions and for extended periods of time.

Moreover, baclofen undergoes acidic and basic hydrolysis at very high or very low pH ranges. Therefore, there remains a need to develop a liquid pharmaceutical composition of baclofen for oral administration which is stable at pH ranges of about 3.0-7.0.

Furthermore, there is a need for developing oral liquid formulations of baclofen that allow for dosing flexibility without sedimentation/sticking problems which can cater to the needs of geriatric and pediatric population.

### SUMMARY OF THE INVENTION

It is an object of the present invention to provide a pharmaceutical composition that is suitable for a ready-to-use or ready-to-administer formulation. The ready-to-use or ready-to-administer formulation shall prevent potential side effects that can be elicited by extemporaneous formulations. The liquid formulation should be suitable for formulating a wide variety of drugs. Said formulations shall allow an accurate and precise dosing of the drug contained therein and shall be particularly suitable in the medical treatment of patient groups with swallowing problems such as pediatric or elderly patients. The drug should be stable within said preparation for a long shelf-life.

Thus, the present invention relates to liquid pharmaceutical compositions of baclofen or a pharmaceutically acceptable salt thereof. In an aspect, the invention provides a stable oral pharmaceutical solution comprising (a) a therapeutically effective amount of baclofen; (b) at least one crystallization inhibitor; and (c) at least one pharmaceutically acceptable liquid vehicle, where a concentration of the baclofen is 2 mg/mL or more; the crystallization inhibitor is selected from the group consisting of polyvinylpyrrolidone (PVP), hydroxypropyl methyl cellulose (HPMC), hydroxyethyl cellulose, glycerin, xylitol, and mixtures thereof, and the solution is in the form of a ready-to-use or a ready-to-administer stable solution that is suitable for oral administration to a subject in need thereof, and the solution does not contain cyclodextrin or cyclodextrin derivatives. More specifically, stable liquid pharmaceutical compositions of baclofen at concentrations of 2 mg/mL or more are provided. Preferably, the liquid pharmaceutical compositions are suitable for oral administration, and stable at pH ranges of 3-7 over a variety of storage conditions, including long-term storage for extended periods of time. Methods of treating spasticity using the inventive pharmaceutical compositions are also provided. The invention further relates to various methods for preparing stable liquid pharmaceutical compositions of baclofen. In some embodiments, the viscosity modifier is xanthan gum, the baclofen is present at a concentration of about 5 mg/mL to about 10 mg/mL, the crystallization inhibitor is HPMC in an amount ranging from about 1 mg/mL to about 200 mg/mL, and the solution has a pH between about 3.5 to about 4.5.

In one aspect, liquid pharmaceutical compositions for oral administration are provided, which comprise baclofen, a pharmaceutically acceptable excipient, and at least one pharmaceutically acceptable liquid vehicle, wherein baclofen is present at a concentration of about 2 mg/mL or more, and wherein the liquid pharmaceutical composition is stable. Preferably, a pharmaceutical composition may comprise: (i) about 5 mg/mL of baclofen; (ii) at least one crystallization inhibitor; (iii) at least one viscosity building agent (iv) optionally, at least one pH modifier; (v) at least one sweetener; and (vi) at least one flavoring agent.

In one aspect, a process for the preparation of a stable, liquid pharmaceutical formulation for oral administration comprising baclofen is provided, wherein the process comprises: (i) adding baclofen to heated purified water (65° C.) with continuous stirring, until a clear solution is obtained, (ii) cooling the solution to room temperature; (iii) adding sodium benzoate to the solution obtained in step (ii); (iv) adding hydroxy propyl methyl cellulose (HPMC) to the solution obtained in the step (iv) under stirring until a clear solution is obtained; (v) adding glycerin or xylitol and/or xanthan gum to the solution obtained in step (iv); (vi) optionally, adding a sweeting agent and/or a flavoring agent (e.g., sucralose and grape flavor) to the solution obtained in step (v); and (vii) adjusting the final pH of the solution by using NaOH/HCl and (viii) making up a final volume by using purified water.

In another aspect, a process for the preparation of a stable, liquid pharmaceutical formulation for oral administration comprising baclofen is provided, wherein the process comprises: (i) adding tartaric acid to heated purified water (65° C.) to provide a solution; (ii) adding baclofen to the solution formed in step (i) with continuous stirring, until a clear solution is obtained, and cooling this solution to room temperature; (iii) adding sodium benzoate to the solution formed in step (ii); (iv) adding hydroxy propyl methyl cellulose to the solution formed in step (iii) under stirring until a clear solution is obtained; (v) adding glycerin or xylitol and/or xanthan gum to the solution formed in step (iv); (vi) optionally, adding a sweeting agent and/or a flavoring agent (e.g., sucralose and grape flavour) to the solution formed in step (v); (vii) adjusting the final pH of the solution by using NaOH/HCl and (viii) making up a final volume by using purified water.

Other aspects relate to methods of treatment using the inventive pharmaceutical compositions. Specifically provided is a method for managing, treating or alleviating the signs and symptoms of spasticity resulting from multiple sclerosis or spinal cord damage or spinal cord disease in a patient by orally administering an effective amount of a liquid pharmaceutical composition, according to the invention, to an adult, pediatric or geriatric patient.

Each aspect above may further have one or more of the following additional elements in any combination:

Element 1: wherein baclofen is present at a concentration ranging from 2 mg/mL to 10 mg/mL, preferably about 5 mg/mL.

Element 2: wherein the pharmaceutical composition is selected from the group consisting of an aqueous solution, a syrup, and an elixir.

Element 3: wherein the pharmaceutically acceptable excipient is selected from the group consisting of a crystallization inhibitor, a pH modifier, a sweetening agent, a flavoring agent, a preservative, an antioxidant, a viscosity building agent, a pH adjusting agent, a buffering agent, a coloring agent, a surfactant and mixtures thereof.

Element 4: wherein the pH modifier is an organic acid selected from the group consisting of malic acid, tartaric acid, succinic acid, fumaric acid, maleic acid, gluconic acid, glucuronic acid, and mixtures thereof.

Element 5: wherein the crystallization inhibitor is selected from the group consisting of polyvinylpyrrolidone (PVP), hydroxypropyl methyl cellulose (HPMC), hydroxyethyl cellulose, glycerin, xylitol, and mixtures thereof. Preferably, the crystallization inhibitor is present in an amount ranging from about 1 mg/mL to about 200 mg/mL, more preferably in an amount ranging from about 5 mg/mL to about 120 mg/mL. For example, a weight ratio of the crystallization inhibitor to the baclofen ranges from about 1:1 to 25:1. In some aspects, the crystallization inhibitor is hydroxypropyl methyl cellulose (HPMC), preferably the crystallization inhibitor is hydroxypropyl methyl cellulose (HPMC) having a viscosity less than about 50 cps.

Element 6: wherein the viscosity building agent is selected from the group consisting of hydroxypropyl cellulose, carboxymethyl cellulose, methylcellulose, sodium carboxymethylcellulose, carbomer, xanthan gum, maltodextrin, acacia, tragacanth, and mixtures thereof.

Element 7: wherein the sweetener is a sugar selected from the group consisting of glucose, fructose, sucrose, tagatose, sucralose, maltitol, isomaltose, lactitol, sorbitol, erythritol, trehalose, maltodextrin, polydextrose, and mixtures thereof.

Element 8: wherein the liquid pharmaceutical composition has a pH in the range of about 4 to about 8, preferably between about 3 to about 5, preferably between about 3.0 to about 4.5, and more preferably between about 3.5 to about 4.5.

Element 9: wherein the solution does not contain cyclodextrin or cyclodextrin derivatives.

Element 10: wherein the solution does not contain citric acid, a salt of citric acid, or any combination thereof.

Element 11: wherein the composition is stable for at least 6 months at 40° 0175% RH or 25° 0160% RH.

Element 12: wherein the composition is stable for at least 6 months at 2° C. to 8° C.

Element 13: wherein the level of BRC-A Impurity in the composition is less than about 4% (w/w), preferably less than about 2% (w/w), more preferably less than about 1% (w/w) as measured by HPLC. Preferably, a level of 4-(4-chlorophenyl)-2-pyrrolidinone is less than about 2% (w/w) as measured by HPLC when stored for 3 months at 40° C./75% RH. Preferably, a level of 4-(4-chlorophenyl)-2-pyrrolidinone in the solution is less than about 2% (w/w) as measured by HPLC when stored for 3 months at 25° C./60% RH.

Element 14: wherein the level of unknown impurity in the composition is less than about 0.2% (w/w), preferably less than about 0.15% (w/w), more preferably less than about 0.1% (w/w) as measured by HPLC.

Element 15: wherein the level of total impurities in the composition is less than about 6% (w/w), preferably less than about 4% (w/w), more preferably less than about 2% (w/w) as measured by HPLC.

Element 16: A process for the preparation of a stable, liquid pharmaceutical composition of baclofen, wherein baclofen is dissolved with the aid of heat.

By way of non-limiting examples, exemplary combinations applicable to the embodiments described in this application may include any combination with one or more of elements 1-16, described above.

### DETAILED DESCRIPTION OF THE INVENTION

Unless defined otherwise, all the technical and scientific terms used herein have the same meanings as commonly known by a person skilled in the art. In the case that there is a plurality of definitions for the terms herein, the definitions provided herein will prevail.

As used herein the term “baclofen” refers to baclofen free base or a pharmaceutically acceptable salt, solvate or hydrate thereof. It also includes geometric isomer or a stereoisomer thereof. In certain aspects, baclofen free base may be used. Any crystalline form of baclofen as well as the amorphous form may be used for the preparation of pharmaceutical compositions of the present invention.

The term “pharmaceutically acceptable salt” refers to baclofen salts which are formed with inorganic or organic acids.

The terms “pharmaceutical composition,” “pharmaceutical product,” “pharmaceutical dosage form,” “dosage form,” “pharmaceutical formulation,” etc., refer to a pharmaceutical composition that may be administered to a patient in need of treatment, which may be in any conventional formulation. For example, the term “pharmaceutical composition” as used herein includes an aqueous solution, a syrup, or an elixir.

Within the context of this invention, the term “solution” refers to a mixture of one or more substances dispersed molecularly (i.e., dissolved) in a dissolving liquid medium or vehicle. The solution is preferably homogeneous, in the sense that each API is essentially uniformly distributed and concentrated in the solution. The liquid solution may be viscous (such as syrup) or not. As already mentioned, a liquid solution differs from a suspension which comprises solid particles dispersed throughout a liquid phase in which they are not soluble.

The terms “stable” and “stability” mean that the evolution of the product with time and/or under specific environmental conditions (i.e., temperature, humidity, etc.) has no significant effects on its quality, safety and/or efficacy for a given time period. It can be measured through the formation of degradation products (impurities), variation of pH, appearance (precipitation), microbial growth, and/or color. The term “stable” indicates both chemical and physical stability.

The terms “about” when used along with a numerical variable, generally means the value of the variable and all the values of the variable within a measurement or an experimental error (e.g., 95% confidence interval for the mean) or within a specified value (e.g., ±10%) within a broader range.

The term “pharmaceutically acceptable” substances mean those, which, according to a common medical judgment, are suitable to be in contact with a tissue of a patient without any inappropriate toxicity, irritation, allergic response, etc., have a reasonable balance between advantages and disadvantages, and can be applied to its target use effectively.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The term “room temperature” may be a temperature between 15° C. and 30° C., preferably between 20° C. and 30° C.

The terms “liquid pharmaceutical composition,” refer to a pharmaceutical composition administered to a patient, including an aqueous solution.

The term “ready-to-use,” as used herein, refers to a formulation that does not require constitution or dilution with a prescribed amount of diluent, e.g., purified water or other suitable diluent, before use by the designated route.

The terms “dosage,” “dose unit” or “dose” as used herein means amount of pharmaceutical formulation comprising therapeutically active agent(s) administered at a time.

The term “subject” refers to an animal, including a human or non-human. The terms patient and subject may be used interchangeably herein.

As used herein, “to treat” a condition or “treatment” of the condition is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable.

The term “ready-to-use” as used herein, refers to a formulation that does not require reconstitution or dilution or mixing with a prescribed quantity of liquid diluent, e.g., purified water or any other suitable liquid diluents (For example, but not limited to simple syrup, Ora-plus syrup, Aromatic Elixir, water for injection, 0.9% saline (normal saline), 0.45% saline (half normal saline), 2.5% dextrose/0.45% saline, 5% dextrose solution, Ringer's solution and Ringer's lactate solution), before use by the oral route. The formulation of the present disclosure ready to be administered can be directly administered without the need for any intervening steps of reconstitution and/or dilution or mixing.

The term “ready-to-administer” as used herein, refers to a formulation that does not require any steps or handling or manipulation before administration and can be directly administered orally to the patient.

The terms “ready-to-use” and “ready-to-administer” can be used interchangeably.

The present application relates to stable ready-to-administer (RTA) or ready-to-use (RTU) baclofen compositions suitable for oral administration comprising baclofen and one or more pharmaceutically acceptable excipient.

The present application relates to stable solution pharmaceutical compositions of baclofen or its pharmaceutically acceptable salt or ester thereof, particularly wherein baclofen is present at a concentration of 2 mg/mL or more. In one aspect, a pharmaceutical composition of the present application comprises baclofen or a pharmaceutically acceptable salt thereof, wherein baclofen concentration is about 2 mg/mL to about 20 mg/mL, about 5 mg/mL to about 10 mg/mL, and preferably 5 mg/mL or more. In some aspects, the lower limit may be a baclofen concentration of about 2 mg/mL, about 2.5 mg/mL, about 3 mg/mL, about 3.5 mg/mL, about 4 mg/mL or about 5 mg/mL. In some aspects, the upper limit may be a baclofen concentration of about 5 mg/mL or more, about 7.5 mg/mL or more, or about 10 mg/mL or more.

Any appropriate form of baclofen can be used to prepare oral solutions of the present invention. For example, any crystalline or amorphous form of baclofen may be used in the pharmaceutical compositions of the present application. In other embodiments, the baclofen can be provided as an aqueous or non-aqueous solutions of baclofen, including buffered solutions.

In an embodiment, the present invention provides pharmaceutical compositions comprising baclofen and a pH modifier. In certain aspects, the pH modifier is an organic acid selected from the group consisting of malic acid, tartaric acid, succinic acid, fumaric acid, maleic acid, gluconic acid, glucuronic acid, and mixtures thereof. In another aspect, the pharmaceutical composition of the present invention comprises baclofen and a pH modifier, wherein the composition further comprises additional pharmaceutical acceptable excipients.

In yet another embodiment, the present invention provides pharmaceutical compositions comprising baclofen and a crystallization inhibitor. In certain aspects, the crystallization inhibitor is selected from the group consisting of polyvinylpyrrolidone (PVP K30), hydroxypropyl methyl cellulose (HPMC), polyvinylpyrrolidone/vinylacetate (Kollidon® VA64), hydroxyethyl cellulose, glycerin, xylitol, and mixtures thereof. In another aspect, the pharmaceutical composition of the present invention comprises baclofen and a crystallization inhibitor, wherein the composition further comprises additional pharmaceutical acceptable excipients.

In certain aspects, the crystallization inhibitor may be HPMC, preferably an HPMC having a viscosity of less than about 50 cps, less than about 40 cps, less than about 30 cps, less than about 20 cps, less than about 10 cps or less than about 5 cps. According to the embodiments of the invention, HPMC is available commercially, for example, from the Dow Chemical Company under the trade designation HPMC E3, HPMC E5, and HPMC E15. As used herein, hydroxypropyl methylcellulose E3 refers to hydroxypropyl methylcellulose have a viscosity of about 3 mPas (cps), E5 refers to hydroxypropyl methylcellulose have a viscosity of about 5 (4 to 6) mPas (cps), and E15 refers to hydroxypropyl methylcellulose have a viscosity of about 15 (13-18) mPas (cps). The viscosity for the hydroxypropyl cellulose may be measured in a 2 weight % solution in water at 20° C. with a Ubbelohde tube viscometer.

The amount of the crystallization inhibitor may be present in an amount sufficient to prevent crystallization, e.g., upon storage for 3 months, or 6 months. In some aspects, the amount of the crystallization inhibitor may be from about 1 mg/mL to about 200 mg/mL, preferably about 5 mg/mL to about 120 mg/mL, about 10 mg/mL to about 100 mg/mL, or about 10 mg/mL.

In some aspects, the weight ratio of the crystallization inhibitor to the baclofen is from about 1:1 to 25:1, preferably from about 1:1 to 10:1, from about 1:1 to 5:1, or about 1:1.

In one embodiment, pharmaceutical composition of the present invention comprising baclofen can be formulated at any suitable pH by using any suitable pH adjusting agent. The pH of the pharmaceutical composition is preferably from about 4 to about 7, when measured at room temperature.

In another embodiment, the present invention relates to method of treating signs and symptoms of spasticity in a subject, the method comprising administering the subject an aqueous solution comprising baclofen and other pharmaceutical acceptable excipients.

In yet another embodiment, the present invention relates to method of treating spasticity resulting from multiple sclerosis, particularly for the relief of flexor spasms and concomitant pain, clonus, and muscular rigidity and spasticity due to spinal cord diseases or damage by oral administering the pharmaceutical composition comprising baclofen and other pharmaceutical acceptable excipients.

Preferably, the liquid pharmaceutical composition will be provided in a dosage form that is suitable for oral administration, including but not limited to a solution, syrup, or elixir. The pharmaceutical compositions may be formulated according to conventional pharmaceutical practice.

Generally, the present invention provides stable aqueous baclofen solutions at concentrations higher than the 2.0 mg/mL, and methods of preparing such solutions. In particular, the present invention provides stable aqueous baclofen solutions for oral administration having concentrations greater than about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 and 6.0 mg/mL. The present invention provides stable aqueous baclofen solutions having concentrations less than about 4.0, 4.5, 5.0, 5.5, 6.0, 6.5, 7.0, 7.5, 8.0, 8.5, 9.0, 9.5, 10.0, 10.5, 11.0, 11.5 or 12.0 mg/mL.

In an embodiment, one or more pharmaceutically acceptable excipients comprises crystallization inhibitors, pH modifiers, surfactants, sweetening agents, flavoring agents, preservatives, antioxidants, viscosity building agents, pH adjusting agents, buffering agents, coloring agents, and mixtures thereof.

In an embodiment, pharmaceutical composition comprising baclofen can be formulated at any suitable pH. As noted above, at very high or very low pH values, baclofen undergoes hydrolysis resulting in increased impurity formation. Preferably, pH of pharmaceutical composition can be from about 3 to about 8 when measured at room temperature. Preferably, the pH is about 3.0 to 4.5.

In an embodiment, the invention relates to stable aqueous solutions of baclofen intended for oral administration comprising baclofen at a concentration of about 5 mg/mL and a buffer, wherein the solution has a pH in between 3.0 to 4.5, and wherein the solution is stable for at least 3 months or 6 months at 2° C.-8° C. or 25° C./60% RH or 40° C./25% RH or 40° C./75% RH.

The pharmaceutical compositions of the present invention may contain a pH adjusting agent. Such compounds include, by way of example and without limitation includes acetic acid, glacial; ammonium carbonate; anhydrous; diethanolamine; potassium hydroxide; fumaric acid; sodium bicarbonate; hydrochloric acid; sodium borate; hydrochloric acid, diluted; sodium carbonate; malic acid; trolamine; phosphoric acid; sodium hydroxide; nitric acid; phosphoric acid, diluted; propionic acid; sulfuric acid.

The pharmaceutical compositions of the present invention may optionally contain a buffering agent. The term “buffering agents” as used herein, are agents used to resist change in pH upon dilution or addition of acid or alkali. Such compounds include, by way of example and without limitation, sodium dihydrogen phosphate monohydrate, disodium hydrogen phosphate anhydrous, acetic acid, sodium acetate, adipic acid, benzoic acid, sodium benzoate, monobasic sodium phosphate, dibasic sodium phosphate, disodium hydrogen phosphate dodecahydrate, lactic acid, tris buffer, potassium metaphosphate, potassium phosphate, monobasic sodium acetate, sodium bicarbonate and others known to those of ordinary skill in the art. The concentration of buffering agents in the present invention ranges from 0.001% to 10%, preferably 0.01% to 5.0%, more preferably 0.05% to 1.0%, based on total weight of the composition.

The term “sweetening agents” refers to both bulk (caloric) and intense (non-caloric) sweeteners, which impart sweet taste to the preparation. Examples of bulk sweeteners are dextrose, fructose, glucose, hydrogenated glucose syrup, isomalt, maltitol, maltose, mannitol, sorbitol, sucrose, xylitol, ribose, deoxyribose, neuraminic acid and mixtures thereof. Examples of intense sweeteners are acesulfame, alitame, aspartame, cyclamate, dihydrochalcone sweetener, neotame, saccharin, sucralose, the pharmaceutically acceptable salts thereof such as sodium or calcium saccharin, acesulfame potassium or sodium cyclamate, and mixtures thereof. In one embodiment, the pharmaceutically acceptable sweetener in the present invention is sucralose. In a preferred embodiment, the concentration of sweetener ranges from 0.001% to 10%, preferably 0.01% to 5.0%, more preferably 0.05% to 1.0%, based on total weight of the composition.

The term “flavouring agent,” as used herein, refers to an agent or a mixture of agents that adds flavor to a mixture. The flavouring agent is selected from the group consisting of a natural flavor, an artificial flavor, and mixtures thereof. Flavoring agents include, but are not limited to, mint, peppermint, cola, apple, vanilla, orange, peach, apricot, raspberry, cherry, honey, lemon, coconut, pineapple, strawberry, grape, banana, mixed berry, mixed red fruit and cream flavors and mixture thereof. In particular, the flavoring agent of the present invention is mixed berry flavor. The concentration of flavoring agent ranges from 0.001% to 10%, preferably 0.01% to 5.0%, more preferably 0.05% to 1.0%, based on total weight of the composition.

The pharmaceutical compositions of the present invention may additionally contain an antioxidant. The term “antioxidant” as used herein, refers to an agent which inhibits oxidation and thus is used to prevent the deterioration of preparations by the oxidative process. Such compounds include by way of example and without limitation, sodium bisulfate, ascorbic acid, ascorbyl palmitate, glycine, L-cysteine hydrochloride, L-methionine, butylated hydroxy anisole, butylated hydroxytoluene, hydro phosphorous acid, monothioglycerol, propyl gallate, sodium ascorbate, sodium sulfide, sodium sulfite, sodium bisulfite, sodium formaldehyde sulfoxylate, thioglycolic acid, sodium metabisulfite, alpha-tocopherol and others known to those of ordinary skill in the art. The concentration of antioxidant in the present invention ranges from 0.001% to 10%, preferably 0.01% to 5.0%, more preferably 0.05% to 1.0%, based on total weight of the composition.

In an embodiment, suitable viscosity building agents (also referred to as viscosity modifiers) include hydroxypropyl cellulose, carboxymethyl cellulose, sodium hydroxypropyl methylcellulose, methylcellulose, methyl ethyl cellulose, sodium carboxymethylcellulose, carbomer, xanthan gum, maltodextrin, acacia, tragacanth and polyvinyl alcohol and mixtures thereof.

In an embodiment, suitable preservatives include anti-microbials, antioxidants, and agents that enhance sterility. Exemplary preservatives include, ascorbyl palmitate, benzyl alcohol, butylated hydroxyanisole (BHA), butylated hydroxytoluene (BHT), erythorbic acid, fumaric acid, malic acid, propyl gallate, sodium ascorbate, sodium benzoate, sodium bisulfate, sodium metabisulfite, sodium sulfite, sodium propionate, parabens (methyl-, ethyl-, propyl-, and butyl-), benzoic acid, potassium sorbate, and vanillin.

In an embodiment, pharmaceutical composition of the present invention comprises baclofen, a crystal growth inhibitor, and a preservative, wherein the concentration of preservative ranges from about 0.1 mg/mL to about 10 mg/mL, preferably about 5 mg/mL.

Any appropriate form of baclofen can be used to prepare oral solutions of the present invention. For example, any crystalline or amorphous form of baclofen may be used in the pharmaceutical composition of the present application. In preferred embodiments, appropriate forms of baclofen include baclofen solids such as powdered, lyophilized, spray-dried, hot-melt extruded or micro-fluidized baclofen. In other embodiments, the baclofen can be provided as an aqueous or non-aqueous solution of baclofen, including buffered solutions.

The pharmaceutical compositions of present application may be filled into any suitable pharmaceutically acceptable containers. For example, the pharmaceutically acceptable container may be selected from group consisting of bottles and syringes.

The bottle can be made of any material convenient with the storage and the use requirements comprising polymers, metal, and glass and so on. It is of importance that the bottle material does not interfere with the components of the liquid formulation as disclosed herein. In an embodiment it is made of glass. In order to protect the APIs from light-induced degradation, a preferred embodiment comprises amber glass bottle.

The bottle capacity can be adapted to the volume to be administrated for the period during which the liquid formulation as disclosed herein is stable. For instance, a solution which is stable for 10 days after opening associated to an administration of two doses of 5 mL per day may be stored into bottle of about 100 mL. The one skilled in the art will easily adapt the volume of the bottle to that needed as previously suggested.

The pipette is made of glass, plastic, or any material convenient with the use and the storage of the liquid solutions as disclosed herein. The pipette may be graduated to facilitate the administration of the liquid solution. In an embodiment, the pipette is a 5 mL graduated pipette.

The cap (or closure) is any article for closing a suitably shaped opening. It encompasses, but is not limited to, childproof closures, waterproof closures, pipette-associated caps, solid caps, plastic, or polymeric caps. In an embodiment, the cap is screwed on the bottle top or interlocked with the top of the bottle.

A sealing element may be required for the tightness of the system bottle-cap or bottle-pipette-cap or bottle-pipette or pipette-cap. This element can be supplied on its own and further fit in the bottle-neck, or around the pipette, or in the cap, or it can be previously adapted to the bottle, the cap, or the pipette.

The invention also relates to a kit of parts comprising a package containing bottles of the liquid formulation as disclosed herein and pipettes intended to remove the needed amount of the liquid formulation and/or instructions.

In another aspect, the invention relates to a kit of parts allowing the extemporaneously preparation of the solutions according to the invention.

In an embodiment, the pharmaceutically acceptable container may be a bottle, wherein the bottle was selected from group consisting of a glass bottle and a plastic bottle. Examples of glass bottle include, but are not limited to Type I, II and III borosilicate glass bottles. In an embodiment, the pharmaceutically acceptable container was a glass bottle, wherein the glass bottle may be amber colour glass bottle or clear glass bottle. Examples of plastic bottles include, but are not limited to, high-density polyethylene (HDPE), polyethylene terephthalate (PET) and polypropylene (PP) bottles. In an embodiment, the pharmaceutically acceptable container is a plastic bottle, wherein the plastic bottle may be amber colour, white opaque or translucent plastic bottle. In preferred embodiment, the HDPE bottles will be available in 30, 60, 120, 250-& 500 ml fill volumes.

In an embodiment, the pharmaceutical composition of present application was packed in a kit comprising bottle with child resistant cap, dosing syringe, adapter, and dosing syringe.

### Stability

As used herein, the term “stable” is defined as no more than about 5% loss of baclofen under typical commercial storage conditions. In certain embodiments, the formulations of the present invention will have no more than about 3% loss of baclofen, more preferably, no more than about 2% loss of baclofen, under typical commercial storage conditions. The composition retains at least about 95% of the potency of baclofen after storing the composition at 40° C./25% RH or 40° C./75% RH for at least three months. In certain aspects, the term “stable” refers to chemical stability, wherein not more than 6.0% w/w of total related substances are formed on storage at accelerated conditions of stability at 40° C./25% RH or 40° C./75% RH or at 25° C. and 60% relative humidity or 2-8° C. for a period of at least six months or to the extent necessary for use of the composition.

In particular, the BRC-A (Baclofen related compound-A) Impurity (i.e., 4-(4-Chlorophenyl)-2-pyrrolidinone) may be monitored. The structure of BRC-A Impurity is shown below:

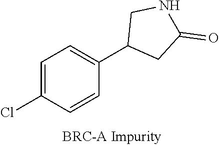 [SMILES: O=C1CC(c2ccc(Cl)cc2)CN1]

Compositions of the present application were found to remain in solution, without any recrystallization or precipitation, when stored for 6 months at 2-8° C., 25° C./60% relative humidity (RH) conditions, or 40° C./25% RH or, 40° C./75% RH conditions.

In an embodiment, the pharmaceutical compositions of the present application were subjected to freeze-thaw cycle testing to determine stability, phase separation or precipitation or crystallization under high and low temperature conditions.

Freeze-thaw testing is conducted by exposing the product to freezing temperatures (−20° C.) for at least 24 hours, and then allowing it to thaw at room temperature for at least 24 hours. This complete cycle is referred to as one Freeze-thaw cycle. Samples were subjected from 1 to 7 Freeze-thaw cycles. The sample was analyzed visually for particles or crystals.

In an embodiment, the invention relates to stable aqueous solutions of baclofen intended for oral administration comprising about 5 mg/mL baclofen and a crystal growth inhibitor, wherein the solutions are stable for at least 3 months or 6 months at 2-8° C. or 25° C./60% RH, or 25° C./40% RH or 30° C./65% RH, or 40° C./25% RH or 40° C./75% RH.

In an embodiment, the invention relates to stable aqueous solutions of baclofen intended for oral administration comprising about 5 mg/mL baclofen, a crystal growth inhibitor and a viscosity building agent, wherein the solutions are stable for at least 3 months or 6 months at 2-8° C. or 25° C./60% RH or 25° 0140% RH or 30° C./65% RH or 40° C./25% RH or 40° C./75% RH.

In yet another embodiment, the invention relates to stable aqueous solutions of baclofen intended for oral administration comprising about 5 mg/mL baclofen and a crystal growth inhibitor, wherein the solutions when stored for at least 3 months at 2° C.-8° C. or 25° C./60% RH or 25° C./40% RH or 30° C./65% RH or 40° C./25% RH or 40° C./75% RH condition exhibits less than about 2% (w/w), preferably less than 1.5% (w/w), more preferably less than 1% (w/w) of BRC-A Impurity as measured by HPLC.

In yet another embodiment, the invention relates to stable aqueous solutions of baclofen intended for oral administration comprising about 5 mg/mL baclofen and a crystal growth inhibitor, wherein the solutions when stored for at least 3 months at 2° C.-8° C. or 25° C./60% RH or 25° C./40% RH or 30° C./65% RH, or 40° C./25% RH, or 40° C./75% RH condition exhibits less than about 0.2% (w/w), preferably less than 0.15% (w/w), more preferably less than 0.1% (w/w) of unknown Impurity (at RRT 0.62) as measured by HPLC.

In another embodiment, the invention relates to stable aqueous solutions of baclofen intended for oral administration comprising about 5 mg/mL baclofen and HPMC, wherein the solutions when stored for at least 6 months at 2-8° C. exhibits less than about 1% (w/w) of BRC-A impurity as measured by HPLC.

In another embodiment, the invention relates to stable aqueous solutions of baclofen intended for oral administration comprising about 5 mg/mL baclofen and polyvinylpyrrolidone, wherein the solutions when stored for at least 12 months at 2-8° C. exhibits less than about 2% (w/w), preferably less than 1.5% (w/w), more preferably less than about 1% (w/w) of BRC-A impurity as measured by HPLC.

In an embodiment, the invention relates to stable aqueous solution of baclofen intended for oral administration comprising about 5 mg/mL baclofen, HPMC and xanthan gum, wherein pH of the aqueous solution is in between 3-7.

In an embodiment, the invention relates to stable aqueous solutions of baclofen intended for oral administration, comprising about 5 mg/mL baclofen and about 10 mg/mL of HPMC.

### Dosage and Administration

The pharmaceutical compositions as described herein may be used in methods of treatment, in which an effective amount of baclofen or a pharmaceutically acceptable salt thereof is administered to a patient.

For administration to animal or human subjects, the pharmaceutical compositions comprise a therapeutically effective dosage amount of baclofen or a pharmaceutically acceptable salt thereof. The formulation may be prepared using conventional methods, for example, depending on the subject to be treated, the mode of administration, and the type of treatment desired (e.g., prevention, prophylaxis, or therapy).

Determination of baclofen optimal dosage may require individual titration. Therapy may be started at a low dosage, and increased gradually until an optimum effect is achieved (e.g., usually between 40-80 mg daily). In certain embodiments, 1-50 mL of baclofen oral solution may be administered to achieve optimum effect, preferably 3-20 mL may be administered to achieve optimum effect.

In an embodiment, the present application relates to method of treating signs and symptoms of spasticity resulting from multiple sclerosis in adult patient, the method comprising administering 5 mg three times a day for 3 days, or 10 mg three times a day for 3 days, 15 mg three times a day for 3 days, or 20 mg three times a day for 3 days, or additional increases as may be necessary up to the maximum recommended dosage of 80 mg daily (20 mg four times a day) to the subject a pharmaceutical composition comprising baclofen or its pharmaceutically acceptable salts thereof.

In an embodiment, the present application relates to method of treating signs and symptoms of spasticity resulting from multiple sclerosis in patients of age less than 18 years with a dose from 0.3 mg/kg a day to 2.5 mg/kg a day, in 2 to 4 divided doses to the subject a pharmaceutical composition comprising baclofen or its pharmaceutically acceptable salts thereof.

In certain aspects, the pharmaceutical compositions described herein may be used to treat adults and adolescents (e.g., about 13-17 years). In certain aspects, the pharmaceutical compositions described herein may be used as monotherapy or as adjunctive therapy. For example, additional active agents may be used in adjunctive therapy with baclofen, such as pain medications (e.g., morphine, hydromorphone, etc.).

The dosage levels can be dependent on the nature of the condition, drug efficacy, the condition of the patient, the judgment of the practitioner, and the frequency and mode of administration. The unit dosage forms can be administered to achieve any daily amount described herein, such as by administering one to five times daily (e.g., one, two, three, four, or five times daily).

By “effective” (also referred to herein as “therapeutically effective”) amount is meant the amount of a drug sufficient to treat, prevent, or ameliorate a condition in a subject or patient. The effective amount of baclofen or pharmaceutically acceptable salt thereof, used to practice the present invention for therapeutic management of a condition may be determined and adjusted by a person of ordinary skill to provide the appropriate amount and dosage regimen, e.g., depending upon one or more of the manner of administration, the age, body weight, sex, and/or general health of the patient.

### General HPLC Procedure

As explained in detail below, the following HPLC procedure can be used to identify assay of baclofen. The materials and general conditions are listed below:

### Chromatographic Conditions

TABLE 1

| Column | Inertsil ODS-HL, 150 × 4.6 mm, 5μ | |
|---|---|---|
| Column Temperature | 30° | C. |
| Sample temperature | 25° | C. |
| Flow rate | 1.0 | mL/min |
| Detector | 220 nm with PDA/UV detector | |
| Injection volume | 10 | μL |
| Run time | 20 | minutes |

| | |
|---|---|
| Mobile Phase A | Dissolve 2.72 g of Potassium dihydrogen |
| | phosphate in 1000 mL of water, adjust the |
| | pH of the solution to 3.20 ± 0.05 with |
| | O-phosphoric acid. |
| Mobile Phase B | Prepare a mixture of acetonitrile, methanol and |
| | water in 70:20:10% V/V/V ratio. |

### Gradient Program

TABLE 2

| Time (min) | % Mobile phase-A | % Mobile phase-B |
|---|---|---|
| 0 | 80 | 20 |
| 3.0 | 70 | 30 |
| 12.0 | 40 | 60 |
| 13.0 | 20 | 80 |
| 16.0 | 20 | 80 |
| 17.0 | 80 | 20 |
| 20.0 | 80 | 20 |

The following HPLC procedure can be used to detect and quantify impurities of baclofen. The materials and general conditions are listed below:

### Chromatographic Conditions

TABLE 3

| Column | Sunfire C18, 250 × 4.6 mm, 5μ | |
|---|---|---|
| Column Temperature | 30° | C. |
| Sample temperature | 25° | C. |
| Flow rate | 0.8 | mL/min |
| Detector | 220 nm with PDA/UV detector | |
| Injection volume | 10 | μL |
| Run time | 70 | minutes |

| | |
|---|---|
| Mobile Phase A | Dissolve 1.38 g of Potassium dihydrogen |
| | phosphate in 1000 mL of water, adjust the |
| | pH of solution to 2.5 ± 0.05 with |
| | o-phosphoric acid. |
| Mobile Phase B | Prepare a mixture of acetonitrile and |
| | water 90:10% V/V ratio. |

### Gradient Program

TABLE 4

| Time (min) | % Mobile phase-A | % Mobile phase-B |
|---|---|---|
| 0.01 | 90 | 10 |
| 5 | 90 | 10 |
| 45 | 60 | 40 |
| 50 | 60 | 40 |
| 60 | 20 | 80 |
| 61 | 90 | 10 |
| 70 | 90 | 10 |

### EXAMPLES

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

TABLE 5

| | | | |
|---|---|---|---|
| A | B | C | D |

| | |
|---|---|
| Composition | mg/mL |

| | | | | |
|---|---|---|---|---|
| Baclofen | 5.00 | 5.00 | 5.00 | 5.00 |
| Tartaric Acid | — | — | 1.00 | 1.00 |
| Sodium Benzoate | 2.00 | 2.00 | 2.00 | 2.00 |
| HPMC E5 | 10.00 | 10.00 | 10.00 | 10.00 |
| Glycerine | 200.00 | — | 200.00 | — |
| Xylitol | — | 100.00 | 100.00 | 100.00 |
| Xanthan Gum | 3.50 | 3.50 | — | 2.00 |
| Sucralose | 2.00 | 2.00 | 2.00 | 2.00 |
| Grape Flavour | 1.00 | 1.00 | 1.00 | 1.00 |
| Purified | q.s. to | q.s. to | q.s. to | q.s. to |
| water (q.s.) | 1 mL | 1 mL | 1 mL | 1 mL |
| Final pH | 4.5 | 4.5 | 4.5 | 4.5 |

### Example 1

Manufacturing Process for Composition A & B:
1. The specified quantity of purified water was taken in a suitable container and heated up to 65° C. with continuous stirring.
2. The specified quantity of baclofen was added to the step 1 purified water under stirring until a clear solution formed. The baclofen solution was brought to room temperature.
3. The specified quantity of sodium benzoate was added to the step 2 solution.
4. The specified quantity of HPMC E5 was added the step 3 solution under stirring until a clear solution was formed.
5. The specified quantity of glycerin/xylitol and xanthan gum were added to the step 4 solution with continuous stirring.
6. The specified quantity of sucralose and grape flavor were added to the step 5 solution under stirring until a clear solution was formed
7. The final pH of the solution was adjusted to 4.5 by using NaOH/HCl.
8. The final volume of the step 7 solution was made up to 1 ml by using purified water.

Manufacturing Process for Composition C & D:
1. The specified quantity of purified water was taken in a suitable container and heated up to 65° C. with continuous stirring.
2. The specified quantity of tartaric acid was added to the step 1 purified water under stirring until a clear solution was formed.
3. The specified quantity of baclofen was added to the step 2 purified water under stirring until a clear solution was formed. The baclofen solution was brought to room temperature.
4. The specified quantity of sodium benzoate was added to the step 3 solution.
5. The specified quantity of HPMC E5 was added the step 4 solution under stirring until a clear solution was formed.
6. The specified quantity of glycerin and/or xylitol and xanthan gum were added to the step 5 solution with continuous stirring. Xanthan gum was not added in the composition C.
7. The specified quantities of sucralose and grape flavor were added to the step 6 solution under stirring until a clear solution was formed
8. The final pH of the solution was adjusted to 4.5 by using NaOH/HCl.
9. The final volume of the step 8 solution was made up to 1 ml by using purified water.

Compositions A, B, C & D were subjected to freeze thaw cycling at −20° C. for 24 hours followed by at room temperature for 24 hours and observed for any visible particles or crystals by physical observation. After 3 freeze thaw cycles, the baclofen in compositions A, B, C & D remained solubilized and the compositions were clear without any recrystallization or precipitation. Stability data of the compositions are summarized in Tables 6-7.

TABLE 6
Stability data of compositions A & B

| | Test | | | | | |
|---|---|---|---|---|---|---|
| | Composition A | | | Composition B | | |
| | Condition | | | | | |
| | | 40° C./75% RH | | | 25° C./60% RH | 40° C./75% RH |
| | Period | | | | | |
| | Initial | 3 months | 6 months | Initial | 3 months | |
| Description | No crystal growth observed | | | | | |
| Assay | 97.4 | 96.4 | 100.6 | 97.3 | 97.0 | 96.5 |

Related substances % w/w

| | | | | | | |
|---|---|---|---|---|---|---|
| BRC-A | 0.09 | 1.14 | 1.12 | 0.07 | 0.13 | 0.42 |
| impurity | | | | | | |
| Maximum | 0.04 | 0.17 | 0.24 | 0.01 | 0.01 | 0.04 |
| unknown | | | | | | |
| Impurity | | | | | | |
| Total Impurities | 0.20 | 1.61 | 1.78 | 0.09 | 0.18 | 0.54 |

TABLE 7
Stability data of compositions C & D

| | Test | | | | | |
|---|---|---|---|---|---|---|
| | Composition C | | | Composition D | | |
| | Condition | | | | | |
| | | 40° C./75% RH | | | 25° C./60% RH | 40° C./75% RH |
| | Period | | | | | |
| | Initial | 3 months | 6 months | Initial | 3 months | |
| Description | No crystal growth observed | | | | | |
| Assay | 106.2 | 106.8 | 105.7 | 104.7 | 104.5 | 105.1 |

Related substances % w/w

| | | | | | | |
|---|---|---|---|---|---|---|
| BRC-A | 0.03 | 0.69 | 1.28 | 0.07 | 0.37 | 1.03 |
| impurity | | | | | | |
| Maximum | 0.09 | 0.18 | 0.18 | 0.01 | 0.03 | 0.05 |
| unknown | | | | | | |
| Impurity | | | | | | |
| Total | 0.14 | 1.32 | 1.99 | 0.08 | 0.46 | 1.21 |
| Impurities | | | | | | |

### Example 2

TABLE 8

| | | | |
|---|---|---|---|
| E | F | G | H |

| | |
|---|---|
| Composition | mg/mL |

| | | | | |
|---|---|---|---|---|
| Baclofen | 5.00 | 5.00 | 5.00 | 5.00 |
| Tartaric Acid | 1.00 | 1.00 | 1.00 | 1.00 |
| Sodium Benzoate | 2.00 | 2.00 | 2.00 | 2.00 |
| HPMC E5 | 10.00 | — | 10.00 | — |
| Xylitol | 100.00 | 100.00 | — | — |
| Xanthan Gum | — | 3.50 | 3.50 | 3.50 |
| Sucralose | 2.00 | 2.00 | 2.00 | 2.00 |
| Grape Flavour | 2.00 | 1.00 | 1.00 | 2.00 |
| Purified water | q.s. to | q.s. to | q.s. to | q.s. to |
| | 1 mL | 1 mL | 1 mL | 1 mL |
| Final pH | 4.2 | 4.5 | 4.5 | 4.5 |

Manufacturing Process of Composition E, F, G & H:
1. The specified quantity of purified water was taken in a suitable container and heated up to 65° C. with continuous stirring.
2. The specified quantity of tartaric acid was added to the step 1 purified water under stirring until a clear solution was formed.
3. The specified quantity of baclofen was added to the step 2 purified water under stirring until a clear solution was formed. The baclofen solution was brought to room temperature.
4. The specified quantity of sodium benzoate was added to the step 3 solution.
5. The specified quantity of HPMC E5 was added the step 4 solution under stirring until a clear solution was formed.
6. The specified quantity of xylitol and/or xanthan gum were added to the step 5 solution with continuous stirring.
7. The specified quantities of sucralose and grape flavor were added to the step 6 solution under stirring until a clear solution was formed
8. The final pH of the solution was adjusted by using NaOH/HCl.
9. The final volume of the step 8 solution was made up to 1 ml by using purified water.

Compositions E, F, G & H were subjected to freeze thaw cycling at −20° C. (24 hours) followed by at room temperature for 24 hours and observed for any visible particles or crystals formation. It was observed that, compositions E, F, G & H were precipitated after first freeze thaw cycle.

### Example 3

TABLE 9

| | I | J | K | L | M |
|---|---|---|---|---|---|
| Composition | mg/mL | | | | |
| Baclofen | 5.00 | 5.00 | 5.00 | 5.00 | 5.00 |
| Tartaric Acid | 1.00 | 1.00 | 1.00 | 1.00 | 1.00 |
| Sodium Benzoate | 2.00 | 2.00 | 2.00 | 2.00 | 2.00 |
| HPMC E5 | 10.00 | 10.00 | 10.00 | 10.00 | 10.00 |
| Xylitol | 100.00 | 100.00 | 100.00 | 100.00 | 100.00 |
| Xanthan Gum | 3.50 | 3.50 | 3.50 | 3.50 | 3.50 |
| Sucralose | 2.00 | 2.00 | 2.00 | 2.00 | 2.00 |
| Grape Flavour | 1.00 | 1.00 | 1.00 | 1.00 | 1.00 |
| Purified | q.s. to | q.s. to | q.s. to | q.s. to | q.s. to |
| water | 1 mL | 1 mL | 1 mL | 1 mL | 1 mL |
| Final pH | 3.5 | 4.0 | 4.5 | 5.0 | 5.5 |

Manufacturing Process of Composition I, J, K, L & M:
1. The specified quantity of purified water was taken in a suitable container and heated up to 65° C. with continuous stirring.
2. The specified quantity of tartaric acid was added to the step 1 purified water under stirring until a clear solution was formed.
3. The specified quantity of baclofen was added to the step 2 purified water under stirring until a clear solution was formed. The baclofen solution was brought to room temperature.
4. The specified quantity of sodium benzoate was added to the step 3 solution.
5. The specified quantity of HPMC E5 was added to the step 4 solution under stirring until a clear solution was formed.
6. The specified quantity of xylitol and xanthan gum were added to the step 5 solution with continuous stirring.
7. The specified quantities of sucralose and grape flavor were added to the step 6 solution under stirring until a clear solution was formed
8. The final pH of the solution was adjusted by using NaOH/HCl.
9. The final volume of the step 8 solution was made up to 1 ml by using purified water

Compositions I, J, K, L & M were subjected to freeze thaw cycling at −20° C. (24 hours) followed by at room temperature for 24 hours and observed for any visible particles or crystals formation. It was observed that the compositions I, J & K remained solubilized and the compositions were clear without any recrystallization or precipitation after 5 freeze thaw cycles and the compositions L & M were precipitated after first freeze thaw cycle. Stability data of compositions I, J & K are summarized in Tables 10-11.

TABLE 10
Stability data of compositions I & J

| | Test | | | | | |
|---|---|---|---|---|---|---|
| | Composition I | | | Composition J | | |
| | Condition | | | | | |
| | | 25° C./60% RH | 40° C./75% RH | | 25° C./60% RH | 40° C./75% RH |
| | Period | | | | | |
| | Initial | 3 months | | Initial | 3 months | |
| Description | No crystal growth observed | | | | | |
| Assay | 101.9 | 102.5 | 101.6 | 101.6 | 101.8 | 101.8 |

Related substances % w/w

| | | | | | | |
|---|---|---|---|---|---|---|
| BRC-A | 0.04 | 0.17 | 1.16 | 0.04 | 0.13 | 0.85 |
| impurity | | | | | | |
| Maximum | 0.05 | 0.04 | 0.11 | 0.05 | 0.02 | 0.06 |
| unknown | | | | | | |
| Impurity | | | | | | |
| Total | 0.10 | 0.22 | 1.53 | 0.09 | 0.22 | 1.09 |
| Impurities | | | | | | |

TABLE 11
Stability data of composition K

| Test | Composition K | | |
|---|---|---|---|
| Condition | Initial | 25° C./60% | 40° C./75% |
| | | RH | RH |

| | |
|---|---|
| Period | 3 months |

| Description | No crystal growth observed | | |
|---|---|---|---|
| Assay | 98.3 | 102.3 | 101.3 |

Related substances % w/w

| | | | |
|---|---|---|---|
| BRC-A impurity | 0.04 | 0.09 | 0.53 |
| Maximum unknown | 0.06 | 0.01 | 0.04 |
| Impurity | | | |
| Total Impurities | 0.13 | 0.13 | 0.69 |

### Example 4

TABLE 12

| | N | O | P |
|---|---|---|---|
| Composition | mg/mL | | |
| Baclofen | 5.00 | 5.50 | 6.00 |
| Tartaric Acid | 1.50 | 1.00 | 1.00 |
| Sodium Benzoate | 2.00 | 2.00 | 2.00 |
| HPMC E5 | 10.00 | 10.00 | 10.00 |
| Xylitol | 100.00 | 100.00 | 100.00 |
| Xanthan Gum | 3.50 | 3.50 | 3.50 |
| Sucralose | 2.00 | 2.00 | 2.00 |
| Grape Flavour | 2.00 | 2.00 | 2.00 |
| Purified water (q.s.) | q.s. to 1 mL | q.s. to 1 mL | q.s. to 1 mL |
| Final pH | 4.2 | 4.2 | 4.2 |

Manufacturing Process of Composition N, O & P:
1. The specified quantity of purified water was taken in a suitable container and heated up to 65° C. with continuous stirring.
2. The specified quantity of tartaric acid was added to the step 1 purified water under stirring until a clear solution was formed.
3. The specified quantity of baclofen was added to the step 2 purified water under stirring until a clear solution was formed. The baclofen solution was brought to room temperature.
4. The specified quantity of sodium benzoate was added to the step 3 solution.
5. The specified quantity of HPMC E5 was added to the step 4 solution under stirring until a clear solution was formed.
6. The specified quantity of xylitol and xanthan gum were added to the step 5 solution with continuous stirring.
7. The specified quantities of sucralose and grape flavor were added to the step 6 solution under stirring until a clear solution was formed
8. The final pH of the solution was adjusted by using NaOH/HCl.
9. The final volume of the step 8 solution was made up to 1 ml by using purified water.

Compositions N, O & P were subjected to freeze thaw cycling at −20° C. for 24 hours followed by at room temperature for 24 hours and observed for any visible particles or crystals by physical observation. After 3 freeze thaw cycles, the baclofen in compositions N, O & P remained solubilized and the compositions were clear without any recrystallization or precipitation.

TABLE 13

| Test | Composition N | | | | |
|---|---|---|---|---|---|
| Condition | Initial | 25° C./ | 40° C./ | 25° C./ | 40° C./ |
| | | 60% | 75% | 60% | 75% |
| | | RH | RH | RH | RH |
| Period | | 3 months | | 6 months | |
| Description | No crystal growth observed | | | | |
| Assay | 102.9 | 103.4 | 101.5 | 103.7 | 103.2 |

Related substances % w/w

| | | | | | |
|---|---|---|---|---|---|
| BRC-A impurity | 0.05 | 0.12 | 0.69 | 0.19 | 1.33 |
| Maximum | 0.01 | 0.01 | 0.05 | 0.02 | 0.10 |
| unknown | | | | | |
| Impurity | | | | | |
| Total Impurities | 0.07 | 0.15 | 0.86 | 0.28 | 1.71 |

### Example 5

TABLE 14

| | | | |
|---|---|---|---|
| Q | R | S | T |

| | |
|---|---|
| Composition | mg/mL |

| | | | | |
|---|---|---|---|---|
| Baclofen | 5.00 | 5.00 | 5.00 | 5.00 |
| Tartaric Acid | 1.00 | 1.00 | 1.00 | 1.50 |
| Sodium Benzoate | 2.00 | 2.00 | 2.00 | 2.00 |
| HPMC E5 | 10.00 | — | — | 10.00 |
| HPMC K4M | 10.00 | — | — | — |
| Kollidon ® VA 64 | — | 10.00 | — | — |
| Hydroxypropyl | — | — | 10.00 | — |
| cellulose | | | | |
| Hydroxyethyl | | | | 3.50 |
| cellulose | | | | |
| Xylitol | 100.00 | 100.00 | 100.00 | 100.00 |
| Xanthan Gum | — | 3.50 | 3.50 | — |
| Sucralose | 2.00 | 2.00 | 2.00 | 2.00 |
| Grape Flavour | 1.00 | 2.00 | 2.00 | 2.00 |
| Purified | q.s. to | q.s. to | q.s. to | q.s. to |
| water (q.s.) | 1 mL | 1 mL | 1 mL | 1 mL |
| Final pH | 4.5 | 4.0 | 4.5 | 4.5 |

Manufacturing Process of Composition Q, R, S & T:
1. The specified quantity of purified water was taken in a suitable container and heated up to 65° C. with continuous stirring.
2. The specified quantity of tartaric acid was added to the step 1 purified water under stirring to form a clear solution.
3. The specified quantity of baclofen was added to the step 2 solution under stirring until a clear solution was formed. The baclofen solution was brought to room temperature.
4. The specified quantity of sodium benzoate was added to the step 3 solution.
5. The specified quantity of HPMC E5 and/or HPMC K4M or Kollidon VA 64 or Hydroxypropyl cellulose or Hydroxyethyl cellulose was added to the step 4 solution under stirring until a clear solution was formed.
6. The specified quantity of xylitol and xanthan gum were added to the step 5 solution with continuous stirring. Compositions Q & T were free of xanthan gum.
7. The specified quantities of sucralose and grape flavor were added to the step 6 solution under stirring until a clear solution was formed
8. The final pH of the solution was adjusted by using NaOH/HCl.
9. The final volume of the step 8 solution was made up to 1 ml by using purified water

Compositions Q, R, S & T were subjected to freeze thaw cycling at −20° C. for 24 hours followed by at room temperature for 24 hours and observed for any visible particles or crystals by physical observation. After 3 freeze thaw cycles, the baclofen in compositions R & T remained solubilized and the compositions were clear without any recrystallization or precipitation, whereas composition Q was precipitated after second cycle and composition S was precipitated after first cycle of freeze thaw cycle.

TABLE 15

| | Test | | | | | |
|---|---|---|---|---|---|---|
| | Composition R | | | Composition T | | |
| | Condition | | | | | |
| | | 25° C./60% RH | 40° C./75% RH | | 25° C./60% RH | 40° C./75% RH |
| | Period | | | | | |
| | Initial | 3 months | | Initial | 3 months | |
| Description | No crystal growth observed | | | | | |
| Assay | 98.6 | 99.7 | 97.6 | 105.9 | 101.6 | 100.9 |

Related substances % w/w

| | | | | | | |
|---|---|---|---|---|---|---|
| BRC-A | 0.03 | 0.12 | 0.54 | 0.33 | 0.09 | 0.43 |
| impurity | | | | | | |
| Maximum | 0.01 | 0.01 | 0.04 | 0.01 | 0.01 | 0.03 |
| unknown | | | | | | |
| Impurity | | | | | | |
| Total | 0.04 | 0.14 | 0.66 | 0.04 | 0.11 | 0.55 |
| Impurities | | | | | | |

### Example 6

TABLE 16

| | U | V | W |
|---|---|---|---|
| Composition | mg/mL | | |
| Baclofen | 5.00 | 5.00 | 5.00 |
| Tartaric Acid | 1.00 | 1.00 | 1.00 |
| Sodium Benzoate | — | — | 2.00 |
| Methyl Paraben | 1.00 | — | — |
| Propyl Paraben | 0.10 | — | — |
| Potassium Sorbate | — | 2.00 | — |
| HPMC E5 | 10.00 | 10.00 | 10.00 |
| Xylitol | 100.00 | 100.00 | 100.00 |
| Xanthan Gum | 3.5 | 3.5 | 1.00 |
| Sucralose | 2.0 | 2.0 | 2.0 |
| Grape Flavour | 2.0 | 2.0 | 1.0 |
| Purified water (q.s.) | q.s. to 1 mL | q.s. to 1 mL | q.s. to 1 mL |
| Final pH | 4.5 | 4.5 | 4.2 |

Manufacturing Process of Composition U, V & W:
1. The specified quantity of purified water was taken in a suitable container and heated up to 65° C. with continuous stirring.
2. The specified quantity of tartaric acid was added to the step 1 purified water under stirring until a clear solution was formed.
3. The specified quantity of baclofen was added to the step 2 purified water under stirring until a clear solution was formed. The baclofen solution was brought to room temperature.
4. The specified quantity of sodium benzoate or potassium sorbate or methyl & propyl paraben was added to the step 3 solution.
5. The specified quantity of HPMC E5 was added to the step 4 solution under stirring until a clear solution was formed.
6. The specified quantity of xylitol and xanthan gum were added to the step 5 solution with continuous stirring.
7. The specified quantities of sucralose and grape flavor were added to the step 6 solution under stirring until a clear solution was formed.
8. The final pH of the solution was adjusted by using NaOH/HCl.
9. The final volume of the step 8 solution was made up to 1 ml by using purified water.

Compositions U, V & W were subjected to freeze thaw cycling at −20° C. for 24 hours followed by at room temperature for 24 hours and observed for any visible particles or crystals by physical observation. After 3 freeze thaw cycles, the baclofen in compositions U, V & W remained solubilized and the compositions were clear without any recrystallization or precipitation.

TABLE 17

| | Test | | | | | |
|---|---|---|---|---|---|---|
| | Composition U | | Composition V | | Composition W | |
| | Condition | | | | | |
| | | 40° C./75% RH | | 40° C./75% RH | | 40° C./75% RH |
| | Period | | | | | |
| | Initial | 3 months | Initial | 3 months | Initial | 3 months |
| Description | No crystal growth observed | | | | | |
| Assay | 102.2 | 99.8 | 101.0 | | 104.4 | 104.5 |

Related substances % w/w

| | | | | | | |
|---|---|---|---|---|---|---|
| BRC-A | 0.04 | 0.53 | 0.04 | ND | 0.05 | 0.38 |
| impurity | | | | | | |
| Maximum | ND | 0.04 | 0.01 | ND | 0.01 | 0.04 |
| unknown | | | | | | |
| Impurity | | | | | | |
| Total | 0.04 | 0.67 | 0.06 | ND | 0.07 | 0.49 |
| Impurities | | | | | | |

Thus, the present invention provides high-concentration, aqueous solutions of baclofen, which are stable under a variety of storage conditions and for extended periods of time.

Having now fully described this invention, it will be understood by those of ordinary skill in the art that it can be performed within a wide equivalent range of parameters without affecting the scope of the invention or any embodiment thereof. All publications, patent applications and patents disclosed herein are incorporated by reference in their entirety.

Unless specified otherwise, all the percentages, portions and ratios in the present invention are on weight basis.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

While compositions and methods are described herein in terms of “comprising” various components or steps, the compositions and methods can also “consist essentially of” or “consist of” the various components and steps.

1. A stable pharmaceutical solution comprising:
a. a therapeutically effective amount of baclofen;
b. at least one crystallization inhibitor; and
c. at least one pharmaceutically acceptable liquid vehicle;
wherein a concentration of the baclofen is 2 mg/mL or more;
wherein the crystallization inhibitor is selected from the group consisting of polyvinylpyrrolidone (PVP), hydroxyethyl cellulose (HEC), xylitol, and mixtures thereof,
wherein the solution is in the form of a ready-to-use or a ready-to-administer stable solution that is suitable for oral administration to a subject in need thereof, and
wherein the solution does not contain cyclodextrin or cyclodextrin derivatives.

2. A stable oral pharmaceutical solution according to claim 1, wherein a level of 4-(4-chlorophenyl)-2-pyrrolidinone is less than about 2% (w/w) as measured by HPLC when stored for 3 months at 40° C./75% RH.

3. The stable oral pharmaceutical solution according to claim 1, wherein the baclofen is present at a concentration of about 5 mg/mL to about 10 mg/mL.

4. The stable pharmaceutical solution according to claim 1, wherein the baclofen is present at a concentration of about 5 mg/mL.

5. The stable pharmaceutical solution according to claim 1, wherein the crystallization inhibitor is present in an amount ranging from about 1 mg/mL to about 200 mg/mL.

6. The stable pharmaceutical solution according to claim 1, wherein the crystallization inhibitor is present in an amount ranging from about 5 mg/mL to about 120 mg/mL.

7. The stable pharmaceutical solution according to claim 1, wherein a weight ratio of the crystallization inhibitor to the baclofen ranges from about 1:1 to 25:1.

8. (canceled)

9. (canceled)

10. The stable pharmaceutical solution according to claim 1, wherein the solution has a pH between about 3.5 to about 4.5.

11. The stable pharmaceutical solution according to claim 1, further comprising one or more pharmaceutically acceptable excipients selected from the group consisting of a pH modifier, a sweetening agent, a flavoring agent, a preservative, an antioxidant, a viscosity modifier, a pH adjusting agent, a buffering agent, a coloring agent, a surfactant, and mixtures thereof.

12. The stable pharmaceutical solution according to claim 1, further comprising a pH modifier selected from the group consisting of maleic acid, gluconic acid, glucuronic acid, and mixtures thereof.

13. The stable pharmaceutical solution according to claim 11, wherein the viscosity modifier is selected from the group consisting of hydroxypropyl cellulose, carboxymethyl cellulose, methylcellulose, sodium carboxymethylcellulose, carbomer, xanthan gum, maltodextrin, acacia, tragacanth, and mixtures thereof.

14. (canceled)

15. (canceled)

16. The stable pharmaceutical solution according to claim 1, wherein the solution is an aqueous solution.

17. The stable pharmaceutical solution according to claim 1, wherein the solution is a non-aqueous solution.

18. The stable pharmaceutical solution according to claim 1, wherein a level of total impurities is less than about 2% (w/w) as measured by HPLC when stored for 3 months at 40° C./75% RH.

19. A method for managing, treating, or alleviating the signs and symptoms of spasticity resulting from multiple sclerosis, spinal cord damage or spinal cord disease in a patient by orally administering a therapeutically effective amount of the stable pharmaceutical solution according to claim 1.

20. The method of claim 19, wherein the patient is a pediatric or a geriatric patient.

21. A stable pharmaceutical solution comprising:
a) 2 mg/mL or more baclofen;
b) at least one crystallization inhibitor; and
c) at least one pharmaceutically acceptable liquid vehicle;
wherein the crystallization inhibitor is selected from the group consisting of polyvinylpyrrolidone (PVP), hydroxypropyl methyl cellulose (HPMC), hydroxyethyl cellulose (HEC), xylitol, and mixtures thereof,
wherein the solution is in the form of a ready-to-use or a ready-to-administer stable solution that is suitable for oral administration to a subject in need thereof,
wherein the solution does not contain cyclodextrin or cyclodextrin derivatives, and
wherein a weight ratio of the crystallization inhibitor to the baclofen ranges from about 1:1 to 25:1.

22. The stable pharmaceutical solution according to claim 21, wherein the crystallization inhibitor is hydroxypropyl methyl cellulose (HPMC) having a viscosity of 3 cps or less.

23. The stable pharmaceutical solution according to claim 22, wherein the stable pharmaceutical solution comprises xanthan gum, the baclofen is present at a concentration of about 5 mg/mL to about 10 mg/mL, the crystallization inhibitor is HPMC in an amount ranging from about 1 mg/m to about 200 mg/mL, and the stable pharmaceutical solution has a pH between about 3.5 to about 4.5.

24. A stable pharmaceutical solution comprising:
a) 2 mg/mL or more baclofen;
b) at least one crystallization inhibitor; and
c) at least one pharmaceutically acceptable liquid vehicle;
wherein the crystallization inhibitor comprises hydroxypropyl methyl cellulose (HPMC) in combination with at least one additional crystallization inhibitor selected from the group consisting of polyvinylpyrrolidone (PVP), hydroxyethyl cellulose (HEC), xylitol, and mixtures thereof,
wherein the solution is in the form of a ready-to-use or a ready-to-administer stable solution that is suitable for oral administration to a subject in need thereof, and
wherein the solution does not contain cyclodextrin or cyclodextrin derivatives.

25. The stable pharmaceutical solution of claim 24, wherein the crystallization inhibitor comprises HPMC and xylitol.
